(19) United States
(12) Patent Application Publication
(10) Pub. No.: US 2011/0281957 A1
(43) Pub. Date: Nov. 17, 2011

(54) ENHANCED BIOACTIVE FORMULATIONS OF RESVERATROL

(75) Inventor: Eric Kuhrts, Bodega, CA (US)

(73) Assignee: Lipoprotein Technologies, Inc., Bodega, CA (US)

(21) Appl. No.: 13/131,579

(22) PCT Filed: Nov. 19, 2009
(86) PCT No.: PCT/US09/65109

§ 371 Date: Aug. 5, 2011

Related U.S. Application Data
(60) Provisional application No. 61/118,354, filed on Nov. 26, 2008.

(51) Int. Cl. A61K 31/05; A61P 3/10; A61P 3/04; A61P 27/02; A61P 3/06; A61P 35/04; A61P 9/00

(52) U.S. Cl. 514/733

(57) ABSTRACT

Methods and formulations for increasing the water solubility and/or bioavailability of resveratrol are disclosed. The formulations may be employed to treat a disease states, including cancer.

### CROSS-REFERENCES TO RELATED APPLICATIONS

This application is a National Stage filing of PCT/US2009/065109, filed Nov. 19, 2009, and claims the benefit of U.S. Provisional Application No. 61/118,354, filed Nov. 26, 2008, which are incorporated by reference herein in their entireties and for all purposes.

### BACKGROUND OF THE INVENTION

Flavonoids are abundant throughout nature and exert a broad range of biological activities in plants and animals. There are now considered to be over 4,000 flavonoids existent in nature. These compounds are the primary source for the colors that occur in the fall in many flowers and trees. Flavonoids are found in fruits, vegetables, nuts, seeds, herbs, spices, stems, flowers, and red wine and tea, and are consumed on a regular basis in the human diet. These compounds can be classified according to their substituents into flavanols, anthocyanidins, flavones, flavanones, and chalcones. Some of the biological activities of flavonoids include; anti-inflammatory, antiviral, antifungal, antibacterial, estrogenic, anti-oxidant, antiallargenic, anticarcinogenic, antithrombotic, hepatoprotective, and antiproliferative medicinal properties.

Some of the principle flavonoids that have been studied and found to have potential as therapeutic agents are; flavonoids or flavanols from green tea and cocoa such as epigallocatechin gallate, epigallocatechin, epicatechin, catechin, and epicatechin gallate, flavonoids from grapes such as resveratrol (3,5,4′-trihydroxystilbene), from soy, such as genistein and diadzein, and quercetin, the richest source of which is onions. Of particular interest has been research related to trans-resveratrol, and certain genes potentially involved in longevity, at least in-vitro and in certain animal models.

Resveratrol and other flavonoids and flavanols have been identified as cancer chemopreventive agents through their interfering action with a variety of cellular mechanisms at low micromolar concentrations such as (1) inhibition of metabolic activation of procarcinogens, (2) induction of carcinogen-detoxifying enzymes, and (3) inhibition of tumor growth by inhibiting inflammatory signals and angiogenesis. Antiproliferative and cytotoxic effects of resveratrol and other flavonoids were tested in breast cancer (MCF-7), colon cancer (HT-29), and ovarian cancer (A-2780) cells in vitro. Resveratrol inhibited the proliferation of MCF-7 and A-2780 cells in a dose-dependent manner. Some flavonoids such as resveratrol can be effective anti-inflammatory agents by inhibition of endogenous prostaglandin synthesis through inhibition of cyclooxygenase (constitutive COX-1 and inducible COX-2) enzymes. Most of the research related to resveratrol has been with trans-resveratrol. Pure trans-resveratrol has recently been available commercially.

In order for any therapeutic molecular substance to be transported through the gastrointestinal tract, enter the blood, and eventually reach the organs and cells inside the body, the molecule must be dissolvable in the aqueous phase of the intestinal fluid. Without dissolution, the drug would pass through the GI-tract as would brick-dust. Flavonoids such as resveratrol are virtually insoluble in water, and animal pharmacokinetic studies of oral doses have demonstrated very low bioavailability. Likewise, human studies with green tea extracts standardized to the active catechins, have demonstrated very low absorption, usually less than 1% of the oral dose in animal or human studies. In fact in one study, plasma tea catechin concentrations determined in humans after oral consumption of a green tea extract were 5-50 times less than the concentrations shown to exert biological activities in in-vitro systems. Animal pharmacokinetic studies with trans-resveratrol also indicate less than 1-2% in plasma after an oral dose. No quercetin could be found in plasma after oral administration of up to 4 g in humans. Many flavonoids are lipophilic or fat soluble, and have very low solubility in water (hydrophobic). Only small amounts of resveratrol are contained in red wine. For example, resveratrol in wine may vary from 0.2 to 5.8 mg/liter. Resveratrol is hydrophobic, or insoluble in water and thus insoluble in water or water containing beverages such as juices or soft drinks.

Due to the many desirable properties of resveratrol, it would be advantageous to have a more water soluble formulation and/or enhanced bioavailability formulation of resveratrol for dosing in-vivo. The present invention solves these and other problems in the art.

### BRIEF SUMMARY OF THE INVENTION

In one aspect, the present invention provides a water-soluble formulation including resveratrol, a resveratrol metabolite or salt thereof, and a non-ionic surfactant.

In another aspect, the present invention provides a method of treating cancer, obesity, diabetes, cardiovascular disease, dyslipidaemia, age-related macular degeneration (e.g. vision loss associated with age-related macular degeneration), high cholesterol, or retinopathy (e.g. diabetic retinopathy) in subject in need of such treatment. The method includes administering to the subject an effective amount of a water soluble formulation disclosed herein.

In another aspect, the present invention provides a method of treating a VEGF-mediated disease state in a subject in need of such treatment. The method includes administering to the subject an effective amount of a water soluble formulation disclosed herein.

In another aspect, the present invention provides a method for enhancing the bioavailability of a resveratrol, a resveratrol metabolite or a salt thereof in a subject. The method includes combining the resveratrol, resveratrol metabolite or salt thereof, and a non-ionic surfactant to form a surfactant-resveratrol mixture. The surfactant-resveratrol mixture may be administered to the subject thereby enhancing the bioavailability of the resveratrol or resveratrol metabolite.

In another aspect, the present invention provides a method of dissolving resveratrol in water. The method includes combining resveratrol, a resveratrol metabolite or salt thereof, with a non-ionic surfactant to form a surfactant-resveratrol mixture. The surfactant-resveratrol mixture is combined with water thereby dissolving the resveratrol in water.

### BRIEF DESCRIPTION OF THE DRAWINGS

Not applicable.

### DETAILED DESCRIPTION OF THE INVENTION

### I. Definitions

The abbreviations used herein have their conventional meaning within the chemical and biological arts.

The term “pharmaceutically acceptable salts” is meant to include salts of the active compounds which are prepared with relatively nontoxic acids or bases, depending on the particular substituent moieties found on the compounds described herein. When formulations of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When formulations of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific formulations of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the formulations of the present invention. Additionally, prodrugs can be converted to the formulations of the present invention by chemical or biochemical methods in an ex vivo environment. For example, prodrugs can be slowly converted to the formulations of the present invention when placed in a transdermal patch reservoir with a suitable enzyme or chemical reagent.

Certain formulations of the present invention can exist in unsolvated forms as well as solvated forms, including hydrated forms. In general, the solvated forms are equivalent to unsolvated forms and are encompassed within the scope of the present invention. Certain formulations of the present invention may exist in multiple crystalline or amorphous forms. In general, all physical forms are equivalent for the uses contemplated by the present invention and are intended to be within the scope of the present invention.

Certain formulations of the present invention possess asymmetric carbon atoms (optical centers) or double bonds; the racemates, diastereomers, tautomers, geometric isomers and individual isomers are encompassed within the scope of the present invention. The formulations of the present invention do not include those which are known in the art to be too unstable to synthesize and/or isolate.

The formulations of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the formulations of the present invention, whether radioactive or not, are encompassed within the scope of the present invention.

“Resveratrol,” as used herein, includes resveratrol derived from natural sources such as grape skins, wine, or other botanical sources such as P. cuspidatum or C. quinquangulata, or produced synthetically as 98% trans-resveratrol, available commercially from Sigma Chemical Co., St. Louis, Mo. Botanical extracts with higher concentrations may be produced by fractionation and further column chromatography until an extract may contain up to a 99% concentration of resveratrol as a mixture of both isomers. “Trans-resveratrol,” as used herein, may also be commonly referred to as trans-3,5,4′-trihydroxystilbene, 3,4′,5-stilbenetriol, (E)-5-(p0hydroxystyryl) resorcinol), and CAS Number 501-36-0.

The term “treating” refers to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the methods of the invention successfully treat a patient's delirium by decreasing the incidence of disturbances in consciousness or cognition.

As used herein, the term “cancer” refers to all types of cancer, neoplasm, or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P₃₈₈ leukemia model is widely accepted as being predictive of in vivo anti-leukemic activity. It is believed that a compound that tests positive in the P₃₈₈ assay will generally exhibit some level of anti-leukemic activity in vivo regardless of the type of leukemia being treated. Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “antineoplastic” means inhibiting or preventing the growth of cancer. “Inhibiting or preventing the growth of cancer” includes reducing the growth of cancer relative to the absence of a given therapy or treatment. Cytotoxic assays useful for determining whether a compound is antineoplastic are well know in the art of cancer therapy and are available for a wide variety of cancers.

As used herein “combination therapy” or “adjunct therapy” means that the patient in need of the drug is treated or given another drug for the disease in conjunction with the formulations of the present invention. This combination therapy can be sequential therapy where the patient is treated first with one drug and then the other or the two drugs are given simultaneously. The present invention includes combination therapy or adjunct therapy using the water soluble formulations of the present invention.

“Patient” refers to a mammalian subject, including human.

As used herein, the term “wet age-related macular degeneration (AMD)” refers to an eye condition or disease in which damaging new blood vessel growth and leakage occurs in the retina, and if left untreated can lead to vision loss. AMD is the leading cause of age related blindness.

As used herein, the term “diabetic retinopathy” refers to an ocular pathology associated with diabetes. Diabetes can cause damage to the blood vessels that nourish the retina, and this can cause the vessels to leak or break, stimulating the growth of abnormal new blood vessels. Diabetic retinopathy is one of the leading causes of blindness in diabetics, and affects more than 4 million adults in America alone.

As used herein, the term “clear aqueous solution” in reference to a solution containing resveratrol means a water containing solution (e.g. a beverage) that is free of visible particles of undissolved resveratrol. In some embodiments, the clear aqueous solution not a dispersion and not a suspension, and remains clear upon sitting undisturbed for 1 hour or more. Where the clear aqueous solution is a beverage, the clear aqueous solution may not need to be shaken prior to consuming.

### II. Water Soluble Formulations

It has been discovered that non-ionic surfactants may be used to increase the solubility and/or bioavailability of resveratrol, resveratrol metabolites, and salts thereof (e.g. a pharmaceutically acceptable salt). Thus, non-ionic surfactants may be used to form water soluble formulations containing resveratrol, a resveratrol metabolite, or salt thereof. In some embodiments, the resveratrol is trans-resveratrol, a trans-resveratrol, or salt thereof. In other embodiments, the resveratrol is trans-resveratrol.

In one aspect, the present invention provides a water-soluble formulation including resveratrol, a resveratrol metabolite or salt thereof, and a non-ionic surfactant. In some embodiments, the water soluble formulation does not include a vegetable oil suspension or visible macro-micelles (micelles visible to the naked eye) in water. In other embodiments, the water soluble formulation does not include an alcohol (e.g. the resveratrol is not first dissolved in alcohol and then added to water).

A “non-ionic surfactant,” as used herein, is a surface active agent that tends to be non-ionized (i.e. uncharged) in neutral solutions (e.g. neutral aqueous solutions). Useful non-ionic surfactants include, for example, non-ionic water soluble mono-, di-, and tri-glycerides; non-ionic water soluble mono- and di-fatty acid esters of polyethyelene glycol; non-ionic water soluble sorbitan fatty acid esters (e.g. sorbitan monooleates such as SPAN 80 and TWEEN 20 (polyoxyethylene 20 sorbitan monooleate)); polyglycolyzed glycerides; non-ionic water soluble triblock copolymers (e.g. poly(ethyleneoxide)/poly-(propyleneoxide)/poly(ethyleneoxide) triblock copolymers such as POLOXAMER 406 (PLURONIC® F-127), and derivatives thereof.

Examples of non-ionic water soluble mono-, di-, and tri-glycerides include propylene glycol dicarpylate/dicaprate (e.g. MIGLYOL® 840), medium chain mono- and diglycerides (e.g. CAPMUL® and IMWITOR® 72), medium-chain triglycerides (e.g. caprylic and capric triglycerides such as LAVRAFAC, MIGLYOL® 810 or 812, CRODAMOL™ GTCC-PN, and SOFTISON 378), long chain monoglycerides (e.g. glyceryl monooleates such as PECEOL™, and glyceryl monolinoleates such as MAISINE™), polyoxyl castor oil (e.g. macrogolglycerol ricinoleate, macrogolglycerol hydroxystearate, macrogol cetostearyl ether), and derivatives thereof.

Non-ionic water soluble mono- and di-fatty acid esters of polyethyelene glycol include d-α-tocopheryl polyethyleneglycol 1000 succinate (TPGS), poyethyleneglycol 660 12-hydroxystearate (SOLUTOL® HS 15), polyoxyl oleate and stearate (e.g. PEG 400 monostearate and PEG 1750 monostearate), and derivatives thereof.

Polyglycolyzed glycerides include polyoxyethylated oleic glycerides, polyoxyethylated linoleic glycerides, polyoxyethylated caprylic/capric glycerides, and derivatives thereof. Specific examples include LABRAFIL® M-1944CS, LABRAFIL® M-2125CS, LABRASOL®, SOFTIGEN®, and GELUCIRE®.

In some embodiments, the non-ionic surfactant is a polyoxyl castor oil, or derivative thereof. Effective polyoxyl castor oils may be synthesized by reacting either castor oil or hydrogenated castor oil with varying amounts of ethylene oxide. Macrogolglycerol ricinoleate is a mixture of 83% relatively hydrophobic and 17% relatively hydrophilic components. The major component of the relatively hydrophobic portion is glycerol polyethylene glycol ricinoleate, and the major components of the relatively hydrophilic portion are polyethylene glycols and glycerol ethoxylates. Macrogolglycerol hydroxystearate is a mixture of approximately 75% relatively hydrophobic of which a major portion is glycerol polyethylene glycol 12-oxystearate.

In some embodiments, the water soluble formulations includes the resveratrol, resveratrol metabolite or salt thereof, and polyoxyl castor oil to form a transparent water soluble formulation. A “transparent water soluble formulation,” as disclosed herein, refers to a formulation that can be clearly seen through with the naked eye and is optionally colored. In some embodiments, the transparent water soluble formulations do not contain particles (e.g. particles of undissolved resveratrol) visible to the naked eye. In certain embodiments, light may be transmitted through the transparent water soluble formulations without diffusion or scattering. Thus, in some embodiments, the transparent water soluble formulations are not opaque, cloudy or milky-white. Transparent water soluble formulations disclosed herein do not include milky-white emulsions or suspensions in vegetable oil such as corn oil. Transparent water soluble formulations are also typically not formed by first dissolving the resveratrol in alcohol, and then mixed with water.

In some embodiments, the water soluble formulation is a non-alcoholic formulation. A “non-alcoholic” formulation, as used herein, is a formulation that does not include (or includes only in trace amounts) methanol, ethanol, propanol or butanol. In other embodiments, the formulation does not include (or includes only in trace amounts) ethanol.

In some embodiments, the formulation is a non-aprotic solvated formulation. The term “non-aprotic solvated,” as used herein, means that water soluble aprotic solvents are absent or are included only in trace amounts. Water soluble aprotic solvents are water soluble non-surfactant solvents in which the hydrogen atoms are not bonded to an oxygen or nitrogen and therefore cannot donate a hydrogen bond.

In some embodiments, the water soluble formulation does not include (or includes only in trace amounts) a polar aprotic solvent. Polar aprotic solvents are aprotic solvents whose molecules exhibit a molecular dipole moment but whose hydrogen atoms are not bonded to an oxygen or nitrogen atom. Examples of polar aprotic solvents include aldehydes, ketones, dimethyl sulfoxide (DMSO), and dimethyl formamide (DMF). In other embodiments, the water soluble formulation does not include (or includes only in trace amounts) dimethyl sulfoxide. Thus, in some embodiments, the water soluble formulation does not include DMSO. In a related embodiment, the water soluble formulation does not include DMSO or ethanol.

In still other embodiments, the water soluble formulation does not include (or includes only in trace amounts) a non-polar aprotic solvent. Non-polar aprotic solvents are aprotic solvents whose molecules exhibit a molecular dipole of approximately zero. Examples include hydrocarbons, such as alkanes, alkenes, and alkynes.

The water soluble formulation of the present invention includes formulations dissolved in water (i.e. aqueous formulations). In some embodiments, the water soluble formulation forms a transparent water soluble formulation when added to water.

In some embodiments, the water soluble formulation consists essentially of resveratrol (e.g. trans-resveratrol), a resveratrol metabolite or salt thereof, and a non-ionic surfactant. Where a water soluble formulation “consists essentially of” resveratrol (e.g. trans-resveratrol) a resveratrol metabolite or salt thereof, and a non-ionic surfactant, the formulation includes resveratrol (e.g. trans-resveratrol) a resveratrol metabolite or salt thereof, a non-ionic surfactant, and optionally additional components widely known in the art to be useful in neutraceutical formulations, such as preservatives, taste enhancers, buffers, water, etc. A water soluble formulation that “consists essentially of” resveratrol (e.g. trans-resveratrol, a resveratrol metabolite or salt thereof) does not include components that would destroy the novelty and inventiveness of the formulation.

In some embodiments, the water soluble formulation is a water solubilized formulation. A “water solubilized formulation,” as used herein, includes resveratrol, a resveratrol metabolite or salt thereof, a non-ionic surfactant, and water (e.g. a water containing liquid) but does not include organic solvents (e.g. ethanol). In some embodiments, the water solubilized formulation a transparent water soluble formulation.

### III. Methods

In another aspect, the present invention provides a method of treating cancer, obesity, diabetes, cardiovascular disease, dyslipidaemia, age-related macular degeneration (e.g. vision loss associated with age-related macular degeneration), high cholesterol, or retinopathy (e.g. diabetic retinopathy) in subject in need of such treatment. The method includes administering to the subject an effective amount of the water soluble formulations disclosed herein. The term “cancer” is defined in detail above.

In some embodiments, a method of lowering cholesterol in a subject in need of cholesterol lowering therapy is provided. The method includes administering to the subject an effective amount of the water soluble formulations disclosed herein. The cholesterol lowering may be total cholesterol lowering or low density lipoprotein (LDL) lowering.

In another aspect, the present invention provides a method of treating a VEGF-mediated disease state in a subject in need of such treatment. The method includes administering to the subject an effective amount of the water soluble formulations disclosed herein.

In some embodiments, a method is provided for reducing VEGF-mediated vascular permeability and/or abnormal blood vessel growth in the retina of a subject in need of such treatment. The method includes administering to the subject an effective amount of the water soluble formulations disclosed herein.

In other embodiments, a method is provided for treating age-related macular degeneration in a subject in need of such treatment. The method includes administering to the subject an effective amount of the water soluble formulations disclosed herein.

In still other embodiments, a method is provided for treating diabetic macular edema in a subject in need of such treatment. The method includes administering to the subject an effective amount of the water soluble formulations disclosed herein.

Vascular endothelial growth factor (VEGF) is a diffusible protein that is specific to vascular endothelial cells and plays a major role in the regulation of physiological and pathological growth of blood vessels. VEGF promotes the growth of vascular endothelial cells that reside in arteries, veins, and lymphatics, but also has the ability to induce vascular leakage. This permeability enhancing activity is a connecting link between this molecule and other pathological states. For example, VEGF is expressed in the majority of human tumors and plays a critical role in tumor angiogenesis and metastasis. In addition, VEGF is directly involved in the pathological process that leads to the cancer, vision loss associated with age-related macular degeneration (including wet age-related macular degeneration), and retinopathies (such as diabetic retinopathy/diabetic macular edema).

Therefore, in some embodiments, a method of reducing the activity of VEGF is provided. The method may be conducted in vitro or in situ for research purposes by contacting VEGF with the water soluble formulation of the present invention. Alternatively, the activity of VEGF may be reduced in a subject by administering to the subject an effective amount of the water soluble formulation of the present invention.

VEGF inhibition can be measured in-vitro in a suitable cell line such as KOP2.16 endothelial cells, or using other techniques such as the Miles assay.

In another aspect, the present invention provides a method for enhancing the bioavailability of resveratrol or resveratrol metabolite in a subject. The method includes combining resveratrol, a resveratrol metabolite or salt thereof, and a non-ionic surfactant to form a surfactant-resveratrol mixture. The surfactant-resveratrol mixture may be administered to the subject thereby enhancing the bioavailability of the resveratrol or resveratrol metabolite. The bioavailability is enhanced compared to the bioavailability of resveratrol in the absence of non-ionic surfactant.

In another aspect, the present invention provides a method of dissolving resveratrol, a resveratrol metabolite or salt thereof in water. The method includes combining resveratrol, a resveratrol metabolite or salt thereof, with a non-ionic surfactant to form a surfactant-resveratrol mixture. The surfactant-resveratrol mixture is combined with water thereby dissolving the resveratrol in water. The solution may be optionally heated to increase solubility. The heating temperature is typically selected to avoid chemical breakdown of the resveratrol and/or non-ionic surfactant. In some embodiments, the resulting solution is a water soluble formulation or transparent water soluble formulation as described above. For example, the resulting solution may be a water soluble formulation that is a crystal clear solution, with no particles visible to the naked eye.

A subject is an organism that is treated using one of the methods of the present invention. In some embodiment, the subject is a mammalian subject, such as a human or domestic animal.

An effective amount of the water soluble formulation of the present invention is an amount sufficient to achieve the intended purpose of a method of the present invention, such as treating a particular disease state in a subject (e.g. a human subject).

### IV. Dosages and Dosage Forms

The amount of resveratrol adequate to treat a disease (e.g. through modulation of VEGF, COX, cell proliferation), is defined as a “therapeutically effective dose”. The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient and disease or condition treated.

Single or multiple administrations of resveratrol formulations can be administered depending on the dosage and frequency as required and tolerated by the patient. The formulations should provide a sufficient quantity of active agent to effectively treat the disease state. Lower dosages can be used, particularly when the drug is administered to an anatomically secluded site in contrast to administration orally, into the blood stream, into a body cavity or into a lumen of an organ. Substantially higher dosages can be used in topical administration. Actual methods for preparing parenterally administrable resveratrol formulations will be known or apparent to those skilled in the art and are described in more detail in such publications as Remington's, supra. See also Nieman, In “Receptor Mediated Antisteroid Action,” Agarwal, et al., eds., De Gruyter, New York (1987).

In some embodiments, the resveratrol is present in the water soluble formulation at a concentration of at least 1%, 5%, 10%, 20%, 25%, 30%, 35%, 45%, 45%, or 50% by weight. In other embodiments the resveratrol is present in the water soluble formulation at a concentration from 0.01%, 0.1%, 1% to 80%, 5% to 50%, 10% to 35%, or 20% to 25% (by weight). The resveratrol may also be present (e.g. in a beverage formulation) at a concentration from 0.5 to 250 mg per 3.3 fluid oz, or around 25 mg per ml. In other embodiments, the resveratrol is present at a concentration from 0.01 mg/ml to 50 mg/ml. There is a maximum concentration for achieving a crystal clear solution. Concentrations of resveratrol above 20% using polyoxyl 40 castor oil (i.e. macrogoglycerol hydroxystearate) as the surfactant will no longer result in a crystal-clear solution in water. Therefore, for resveratrol or trans-resveratrol, the concentration range would be from 0.1% to 20% in the surfactant, or 0.01 to 20 mg/ml, with the preferred concentration around 20 mg/ml. This represents a ratio of resveratrol to surfactant of 1:5. In some concentrated formulations (e.g. a soft gel capsule formulation), resveratrol may be present at about 1 to 50 mg/ml, or around 20 mg/ml, or at least 1 mg/ml.

In other embodiments, at least 0.5 mg, 1 mg, 2 mg, 3 mg, 4 mg, 5 mg, 10 mg, 20 mg, 30 mg, 40 mg, 50 mg, 100 mg, 200 mg, 300 mg, 400 mg, 500 mg, or 1 g of resveratrol is present in the water soluble beverage formulation. In other embodiments, 0.1 mg to 2g, 0.5 mg to 1 g, 1 mg to 500 mg, 1 mg to 100 mg, 1 mg to 50 mg, 1 mg to 10 mg, or 1 mg to 5 mg of resveratrol is present in the water soluble beverage formulation.

In some embodiments, the water soluble formulation is in the form of a pharmaceutical composition. The pharmaceutical composition may include resveratrol, or resveratrol metabolite, a non-ionic surfactant, and a pharmaceutically acceptable excipient. After a pharmaceutical composition including resveratrol of the invention has been formulated in an acceptable carrier, it can be placed in an appropriate container and labeled for treatment of an indicated condition. For administration of resveratrol, such labeling would include, e.g., instructions concerning the amount, frequency and method of administration. In one embodiment, the invention provides for a kit for the treatment of delirium in a human which includes resveratrol and instructional material teaching the indications, dosage and schedule of administration of resveratrol.

Any appropriate dosage form is useful for administration of the water soluble formulation of the present invention, such as oral, parenteral and topical dosage forms. Oral preparations include tablets, pills, powder, dragees, capsules (e.g. soft-gel capsules), liquids, lozenges, gels, syrups, slurries, beverages, suspensions, etc., suitable for ingestion by the patient. The formulations of the present invention can also be administered by injection, that is, intravenously, intramuscularly, intracutaneously, subcutaneously, intraduodenally, or intraperitoneally. Also, the formulations described herein can be administered by inhalation, for example, intranasally. Additionally, the formulations of the present invention can be administered transdermally. The formulations can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Thus, the formulations described herein may be adapted for oral administration.

For preparing pharmaceutical compositions from the formulations of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

Suitable carriers include magnesium carbonate, magnesium stearate, talc, sugar, lactose, pectin, dextrin, starch (from corn, wheat, rice, potato, or other plants), gelatin, tragacanth, a low melting wax, cocoa butter, sucrose, mannitol, sorbitol, cellulose (such as methyl cellulose, hydroxypropylmethyl-cellulose, or sodium carboxymethylcellulose), and gums (including arabic and tragacanth), as well as proteins such as gelatin and collagen. If desired, disintegrating or co-solubilizing agents may be added, such as the cross-linked polyvinyl pyrrolidone, agar, alginic acid, or a salt thereof, such as sodium alginate. In powders, the carrier is a finely divided solid, which is in a mixture with the finely divided active component. In tablets, the active component is mixed with the carrier having the necessary binding properties in suitable proportions and compacted in the shape and size desired.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (I.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain resveratrol mixed with a filler or binders such as lactose or starches, lubricants such as talc or magnesium stearate, and, optionally, stabilizers. In soft capsules, resveratrol may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, beverages, and emulsions, for example, water or water/propylene glycol solutions. For parenteral injection, liquid preparations can be formulated in solution in aqueous polyethylene glycol solution.

Aqueous solutions and beverages suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending resveratrol in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The formulations of the invention can be delivered transdermally, by a topical route, formulated as applicator sticks, solutions, suspensions, emulsions, gels, creams, ointments, pastes, jellies, paints, powders, and aerosols.

The formulations can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the formulations of the invention are useful for parenteral administration, such as intravenous (IV) administration or administration into a body cavity or lumen of an organ. The formulations for administration will commonly comprise a solution of resveratrol dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of resveratrol in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the resveratrol, resveratrol metabolite or slat thereof into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The formulations may be administered as a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

### V. Assays

Subject non-ionic surfactants may be assayed for their ability to solubilize resveratrol or resveratrol metabolite using any appropriate method. Typically, a non-ionic surfactant is contacted with the resveratrol and mixed mechanically and/or automatically using a shaker or sonicator device. Water may be optionally added, for example, where the resveratrol and/or surfactant is in powder form. The solution may be optionally heated to increase solubility. The heating temperature is selected to avoid chemical breakdown of the resveratrol or resveratrol metabolite and non-ionic surfactant.

The resulting solution may be visually inspected for colloidal particles to determine the degree of solubility of the resveratrol. Alternatively, the solution may be filtered and analyzed to determine the degree of solubility. For example, a spectrophotometer may be used to determine the concentration of resveratrol present in the filtered solution. Typically, the test solution is compared to a positive control containing a series of known quantities of pre-filtered resveratrol solutions to obtain a standard concentration versus UV/vis absorbance curve. Alternatively, high performance liquid chromatography may be used to determine the amount of resveratrol in solution.

High throughput solubility assay methods are well known in the art. Typically, these methods involve automated dispensing and mixing of solutions with varying amounts of non-ionic surfactants, resveratrol, and optionally other co-solvents. The resulting solutions may then be analyzed to determine the degree of solubility using any appropriate method as discussed above.

For example, the Millipore MultiScreen Solubility filter Plate® with modified track-etched polycarbonate, 0.4 μm membrane is a single-use, 96-well product assembly that includes a filter plate and a cover. The device is intended for processing aqueous solubility samples in the 100-300 μL volume range. The vacuum filtration design is compatible with standard, microtiter plate vacuum manifolds. The plate is also designed to fit with a standard, 96-well microtiter receiver plate for use in filtrate collection. The MultiScreen Solubility filter Plate® has been developed and QC tested for consistent filtration flow-time (using standard vacuum), low aqueous extractable compounds, high sample filtrate recovery, and its ability to incubate samples as required to perform solubility assays. The low-binding membrane has been specifically developed for high recovery of dissolved organic compounds in aqueous media.

The aqueous solubility assay allows for the determination of resveratrol solubility by mixing, incubating and filtering a solution in the MultiScreen Solubility filter plate. After the filtrate is transferred into a 96-well collection plate using vacuum filtration, it is analyzed by UV/Vis spectroscopy to determine solubility. Additionally, LC/MS or HPLC can be used to determine compound solubility, especially for compounds with low UV/Vis absorbance and/or compounds with lower purity. For quantification of aqueous solubility, a standard calibration curve may be determined and analyzed for each compound prior to determining aqueous solubility.

Test solutions may be prepared by adding an aliquot of concentrated drug or compound. The solutions are mixed in a covered 96-well MultiScreen Solubility filter plate for 1.5 hours at room temperature. The solutions are then vacuum filtered into a 96-well, polypropylene, V-bottomed collection plate to remove any insoluble precipitates. Upon complete filtration, 160 μL/well are transferred from the collection plate to a 96-well UV analysis plate and diluted with 40 μL/well of acetonitrile. The UV/vis analysis plate is scanned from 260-500 nm with a UV/vis microplate spectrometer to determine the absorbance profile of the test compound.

Thus, one skilled in the art may assay a wide variety of non-ionic surfactants to determine their ability of solubilize resveratrol compounds.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the formulations are equally applicable to the methods of treating disease states described herein. All publications, patents, and patent applications cited herein are hereby incorporated by reference in their entirety for all purposes.

### VI. Examples

The examples below are meant to illustrate certain embodiments of the invention, and are intended to limit the scope of the invention.

Lucifer Yellow was purchased from Molecular Probes (Eugene, Oreg.). Hanks buffer and all other chemicals were obtained from Sigma-Aldrich (St. Louis, Mo.).

### Example 1

Water soluble compositions of trans-resveratrol were formulated containing the non-ionic surfactant macrogolglycerol hydroxystearate (polyoxyl 40 castor oil). By heating and stirring this polyoxyl castor oil with a powdered synthetic 99% trans-resveratrol, a clear viscous solution was formed containing dissolved trans-resveratrol (hereinafter referred to as “resveratrol gel formulation”). The resveratrol gel formulation consisted of macrogolglycerol hydroxystearate 40 (50 ml) and powdered trans-resveratrol (3 grams), representing a concentration of 6% for the resveratrol in the surfactant. The resveratrol/surfactant mixture was slowly added to 100 ml of warm water until a crystal clear solution was formed. To this was added 0.4 g of ascorbic acid to stabilize the trans-resveratrol, as it was noticed in a previous experiment that if the ascorbic acid was not added, the solution began to rapidly change color, turning a dark burgundy to eventually brown color. With the added ascorbic acid, the solution remained clear, with no color change.

As can be seen from the above example, an aqueous solution of solubilized trans-resveratrol was achieved by adding water to the resveratrol gel formulation (hereinafter referred to as “aqueous resveratrol formulation”, or “water soluble beverage”. More specifically, the aqueous resveratrol formulation was prepared by warming the trans-resveratrol gel formulation in warm water to form a “clear aqueous solution” of trans-resveratrol. This aqueous resveratrol formulation did not have undesirable flavor. The aqueous resveratrol formulation consisted of water (100 ml), macrogolglycerol hydroxystearate 40 (50 ml), and powdered 98% trans-resveratrol (3 grams), a concentration of trans-resveratrol in the aqueous resveratrol formulation of 20% (water containing beverage). The aqueous resveratrol formulation was analyzed by HPLC and found to contain 0.2%, or 20 mg/ml trans-resveratrol.

### Example 2

The solubility of the powdered trans-resveratrol in pH 7.4 Hank's Balanced Salt Solution (10 mM HEPES and 15 mM glucose) was compared to the trans-resveratrol gel formulation. At least 1 mg of powdered synthetic trans-resveratrol extract or 100 mg of resveratrol gel formulation was combined with 1 ml of buffer to make a ≧1 mg/ml powdered resveratrol extract mixture and a ≧1 mg/ml resveratrol gel formulation mixture, respectively. The mixtures were shaken for 2 hours using a benchtop vortexer and left to stand overnight at room temperature. After vortexing and standing overnight, the powdered resveratrol extract mixture was then filtered through a 0.45-μm nylon syringe filter (Whatman, Cat# 6789-0404) that was first saturated with the sample.

After vortexing and standing overnight, the resveratrol gel formulation mixture was centrifuged at 14,000 rpm for 10 minutes. The filtrate or supernatant was sampled twice, consecutively, and diluted 10, 100, and 10.000-fold in a mixture of 50:50 assay buffer:acetonitrile prior to analysis.

Both mixtures were assayed by LC/MS/MS using electrospray ionization against the standards prepared in a mixture of 50:50 assay buffer:acetonitrile. Standard concentrations ranged from 1.0 μM down to 3.0 nM. Results are presented in Table 1 below.

TABLE 1
Solubility of Resveratrol in pH 7.4 Phosphate Buffer

| | Solubility (μM) | | |
|---|---|---|---|
| Test Article Identification | Rep 1 | Rep 2 | AVG |
| Powdered Resveratrol | 0.40 | 0.61 | 0.505 |
| Resveratrol Gel | 1680 | 1745 | 1712 |
| Formulation | | | |

As shown in Table 1, the powdered resveratrol extract and resveratrol gel formulation gel showed average solubility values in pH 7.4 Hank's Balanced Salt Solution of 0.5 μM and 1712 μM, respectively.

### Example 3

The permeability of the resveratrol gel through a cell-free (blank) microporous 0.4 micron membrane filter will be studied in order to determine the non-specific binding and cell-free diffusion Pₐₚₚ of the resveratrol gel formulation through the filter. The resveratrol gel formulation will be assayed at the 2 μM resveratrol concentration in Hanks buffer (Hanks Balanced Salt Solution (HBSSg) containing 10 mM HEPES and 15 mM glucose) at a pH of 7.4 in duplicate. Donor samples were collected at 120 minutes. Receiver samples will be collected at 60 and 120 minutes. The apparent permeability coefficient, Pₐₚₚ, and percent recovery are calculated as follows:

Pₐₚₚ=(dCᵣ/dt)×Vᵣ/(A×C₀)

Percent Recovery=100×((Vᵣ×Cᵣ^{final})+(V_{d}×C_{d}^{final}))/(V_{d}×C₀)

Where:
dCᵣ/dt is the slope of the cumulative concentration in the receiver compartment versus time in μM s⁻¹.
Vᵣ is the volume of the receiver compartment in cm³.
V_{d} is the volume of the donor compartment in cm³.
A is the area of the cell-free insert (1.13 cm² for 12-well Transwell).
Cᵣ^{final} is the cumulative receiver concentration in μM at the end of the incubation period.
C_{d}^{final} is the concentration of the donor in μM at the end of the incubation period.
C₀ is the initial concentration of the dosing solution in μM.

Results of the non-specific binding assessment can be presented as in Table 2, which shows the permeability (10⁻⁶ cm/s) and recovery of Resveratrol across the cell-free filter.

TABLE 2

| Resveratrol Dosing Solution | | |
|---|---|---|
| Concentration (μM) | Pₐₚₚ (10⁻⁶ cm/s) | |
| (Average, N = 2) | A-to-B^{A} | Recovery (%)^{B} |
| Rep. 1: | NA | NA |
| Rep. 2: | NA | NA |
| AVERAGE: | AVERAGE: | AVERAGE: |

^{A}A low rate of diffusion (<20 × 10⁻⁶ cm/s) through the cell-free membrane may indicate a lack of free diffusion, which may affect the measured permeability.
^{B}Low recoveries caused by non-specific binding, etc. would affect the measured permeability.

### Example 4

To test the permeability of resveratrol across Caco-2 cell monolayers, Caco-2 cell monolayers will be grown to confluence on collagen-coated, microporous, polycarbonate membranes in 12-well Costar Transwell® plates. Details of the plates and their certification are shown below in Table 3. The test article was will be the aqueous resveratrol formulation, and the dosing concentration would be 2 μM in the assay buffer (HBSSg) as in the previous example. Cell monolayers are dosed on the apical side (A-to-B) or basolateral side (B-to-A) and incubated at 37° C. with 5% CO₂ in a humidified incubator. Samples are taken from the donor chamber at 120 minutes, and samples from the receiver chamber were collected at 60 and 120 minutes. Each determination will be performed in duplicate. Lucifer yellow permeability is also measured for each monolayer after being subjected to the test article to ensure no damage was inflicted to the cell monolayers during the permeability experiment. Permeability of samples of atenolol, propranolol and digoxin are also measured to compare with the permeability of the resveratrol sample. All samples will be assayed for Resveratrol, or corresponding comparative compounds, by LC/MS/MS using electrospray ionization. The apparent permeability (Pₐₚₚ), and percent recovery are calculated as described above. Resveratrol permeability results can be presented as by reporting the permeability (10⁻⁶ cm/s) and recovery of Resveratrol across Caco-2 cell monolayers. All monolayers pass the post-experiment integrity control with Lucifer yellow Papp <0.8×10−6 cm/s.

### Example 5

The following formulation was prepared as described below: 500 mg of purified resveratrol 99% derived from polygonum cuspidatum (Japanese knotweed) was dissolved in 25 ml of warm macrogoglycerol hydroxystearate until a clear gel was formed. 250 mg of ascorbic acid was added to the clear gel, which was then slowly added to 100 ml of warm water. A clear solution was formed with no visible particles or micelles.

What is claimed is:

1. A water-soluble formulation comprising:
a) resveratrol or resveratrol metabolite; and
b) a non-ionic surfactant.

2. The formulation of claim 1, wherein said resveratrol is trans-resveratrol.

3. The formulation of claim 1, wherein said formulation is a non-alcoholic formulation.

4. The formulation of claim 1, wherein said formulation is a non-aprotic solvated formulation.

5. The formulation of claim 1, wherein said resveratrol is present at a concentration of at least 0.01 mg/ml.

6. The formulation of claim 1, wherein said resveratrol is present at a concentration of at least 1 mg/ml.

7. The formulation of claim 1, wherein said resveratrol is present at a concentration of at least 0.01% by weight.

8. The formulation of claim 1, wherein said resveratrol is present at a concentration of at least 20% by weight.

9. The formulation of claim 1, comprising from 1 mg to 25 mg of resveratrol.

10. The formulation of claim 1, comprising at least 10 mg of resveratrol.

11. The formulation of claim 1, wherein said non-ionic surfactant is a non-ionic water soluble mono-, di-, or tri-glyceride; non-ionic water soluble mono- or di-fatty acid ester of polyethyelene glycol; non-ionic water soluble sorbitan fatty acid ester; polyglycolyzed glyceride; non-ionic water soluble triblock copolymers; or derivative thereof.

12. The formulation of claim 1, wherein said non-ionic surfactant is a non-ionic water soluble mono-, di-, or tri-glyceride.

13. The formulation of claim 1, wherein said non-ionic surfactant is polyoxyl 40 castor oil.

14. The formulation of claim 1, wherein said non-ionic surfactant is macrogolglycerol ricinoleate or macrogolglycerol hydroxystearate.

15. The formulation of claim 1, wherein said non-ionic surfactant is macrogolglycerol hydroxystearate.

16. The formulation of claim 1, wherein said formulation is an oral formulation.

17. The formulation of claim 16, wherein said oral formulation is a soft gel capsule.

18. The formulation of claim 16, wherein said oral formulation is a tablet.

19. The formulation of claim 16, wherein said oral formulation is a beverage.

20. The formulation of claim 1, wherein said formulation is an injectable formulation.

21. The formulation of claim 1, wherein said formulation is a topical formulation.

22. The formulation of claim 1, wherein said resveratrol is derived from grapes or polygonum cuspidatum.

23. The formulation of claim 1 further comprising a pharmaceutically acceptable excipient.

24. A method of dissolving resveratrol in water, said method comprising the steps of:
a. combining resveratrol with a non-ionic surfactant to form a surfactant-resveratrol mixture; and
b. combining the surfactant-resveratrol mixture with water thereby dissolving the resveratrol in water.

25. The method of claim 24, wherein said non-ionic surfactant is a polyoxyl 40 castor oil.

26. A method of treating cancer, obesity, diabetes, cardiovascular disease, dyslipidaemia, vision loss associated with age-related macular degeneration, high cholesterol, or diabetic retinopathy in a subject in need of such treatment, said method comprising administering to the subject an effective amount of the formulation of claim 1.

27. A method of treating a VEGF-mediated disease state in a subject in need of such treatment, said method comprising administering to the subject an effective amount of the formulation of claim 1.

28. The method of claim 27, wherein said disease state is vision loss associated with age-related macular degeneration, or diabetic retinopathy.

29. The method of claim 27, wherein said disease state is obesity, diabetes, cardiovascular disease, or dyslipidaemia.

30. A method of enhancing the bioavailability of a resveratrol or resveratrol metabolite in a subject, said method comprising the steps of combining said resveratrol or resveratrol metabolite, and a non-ionic surfactant to form a surfactant-resveratrol mixture.

31. The method of claim 30, further comprising administering said surfactant-resveratrol mixture to said subject thereby enhancing the bioavailability of said resveratrol or resveratrol metabolite.

32. The method of claim 30, wherein the resveratrol is trans-resveratrol.
